(19) United States
(12) Patent Application Publication
(10) Pub. No.: US 2010/0285611 A1
(43) Pub. Date: Nov. 11, 2010

(54) PHOTOBLEACHING RESISTANT PH SENSITIVE DYE NANOREACTORS WITH DUAL WAVELENGTH EMISSION

(76) Inventors: Agnes E. Ostafin, Layton, UT (US); Hiroshi Mizukami, Pasadena, CA (US); Yen-Chi Chen, Salt Lake City, UT (US)

Correspondence Address: THORPE NORTH & WESTERN, LLP., P.O. Box 1219, SANDY, UT, 84091-1219, US

(21) Appl. No.: 12/775,038

(22) Filed: May 6, 2010

Related U.S. Application Data
(60) Provisional application No. 61/175,927, filed on May 6, 2009.

(51) Int. Cl. G01N 33/543; G01N 31/22

(52) U.S. Cl. 436/518; 436/163

(57) ABSTRACT

A pH sensitive nanoreactor can include an aqueous core within a liposome. The aqueous core can include a pH responsive dye dispersed or dissolved within the core. The liposome provides a nanoscale environment for the dye. Further, a nanoshell can be present which encapsulates the liposome. The nanoshell can be permeable to hydrogen ions while also protecting the dye from exposure to deleterious compounds and photobleaching.

### RELATED APPLICATIONS

This application claims the benefit of earlier filed U.S. Provisional Patent Application No. 61/177,737, filed May 13, 2009 which is incorporated herein by reference.

### BACKGROUND OF THE INVENTION

Detection of pH variations at nanoscale resolution poses unique challenges. For instance, in microfluidic devices and biological cells measurement of pH variations could provide insight into function of these devices and the related materials. When the regional dimensions for pH measurement reach the nanoscale, conventional pH detecting methods such as glass electrodes are not usable. Furthermore, the light intensity from small numbers of diffusing pH responsive dye molecules is too low for single or low molecule count detection. The signal averaging is further compromised by the tendency of dye molecules to photobleach under prolonged illumination. The process can also release active photoproducts that affect the surrounding pH levels.

There have been a number of efforts to produce improved intracellular pH sensors. For example, nanoparticles with either fluorescent or surface-enhanced Raman properties have been suggested as alternatives. These include nanosized polyacrylimide PEBBLEs (Probes Encapsulated by Biologically Localized Embedding); Lipobeads; silver nanoparticles; and hollow gold nanoparticles 30 nm in diameter coated with 4-mercaptobenzoic acid which exhibit a surface-enhanced Raman scattering effect (SERS) that is sensitive to pH changes between 6-8. More recently, a two-fluorophore-doped silicate nanoparticle for intracellular pH determination was reported. These particles showed resistance to photobleaching and pH to resolution of only ±0.5 units was recorded, likely due to variations in dye composition.

To achieve this improved resolution in pH measurement, molecules with relatively stable two wavelengths emission appear to be desirable. A dual-wavelength emission dye, carboxy-seminaphtorhodafluor-1 (Carboxy-SNARF-1) is a kind of dye, which shows the shift of emission peak from yellow-orange to deep red fluorescence when the pH changes from acidic to basic conditions. Ratiometric analyses of the dual-emission data, typically at wavelengths of 580 nm and 640 nm, allow the building of pH calibration curves that are independent of changes in dye concentration and in emission intensity. However, the dilution factor can make the dye's fluorescence emission signal too weak to detect, and the emission could be affected by other chromophores and binding to proteins. Others have designed a micron sized polystyrene sphere, modified with pH sensitive fluorochrome Carboxy-SNARF-1 on the surface, as a non-invasive sensor to probe the local changes in pH, within a microfluidic device. Localizing the Carboxy-SNARF-1 molecules on the surface of polystyrene sphere reduced the local dilution factor, creating a point probe that was easily located. However, in this example, the dye was still on the surface, where it can still be affected by quenchers in the surrounding environment.

Therefore, none of the existing techniques provides pH measurement at the nanoscale with useful pH resolution.

### SUMMARY

A pH sensitive nanoreactor can include an aqueous core within a liposome as illustrated in FIG. 1. The aqueous core can include a pH responsive dye dispersed or dissolved within the core. The liposome provides a nanoscale environment for the dye. Further, a nanoshell can be present which encapsulates the liposome. The nanoshell can be permeable to hydrogen ions while also protecting the dye from exposure to deleterious compounds and photobleaching.

The pH sensitive nanoreactors described herein can be particularly effective in measuring pH of nanoscale environments. The pH sensitive nanoreactor can be delivered or otherwise exposed to a nanoscale environment. An emission response of the pH responsive dye can be measured in any number of techniques including measuring emission intensity using a spectrophotometer. The emission intensity can then be correlated to a pH via a predetermined calibration scale.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

The present invention will become more fully apparent from the following description and appended claims, taken in conjunction with the accompanying drawings. Understanding that these drawings merely depict exemplary embodiments of the present invention and they are, therefore, not to be considered limiting of its scope. It will be readily appreciated that the components of the present invention, as generally described and illustrated in the figures herein, could be arranged, sized, and designed in a wide variety of different configurations. Nonetheless, the invention will be described and explained with additional specificity and detail through the use of the accompanying drawings in which:

FIG. 1 is a schematic of a pH nanoreactor in accordance with an embodiment.

FIG. 2 shows C. SNARF-1 structure at pH5 and pH10, respectively.

FIG. 3A is a TEM image of Carboxy-SNARF-1 filled nanoreactors. The outer dark grey shell is made of calcium phosphate, while the inner grey core is liquid filled and contains Carboxy-SNARF-1 dye in accordance with one embodiment of the present invention.

FIG. 3B are dynamic light scattering size distributions of EPC liposomes (labeled as liposome) and calcium phosphate nanoshells prepared using 100 nm extrusion filters in accordance with one embodiment of the present invention.

FIG. 4A-4D illustrate the pH dependent emission spectra of cSNARF-1 in a buffer solution excited at 514 nm and the change of intensity at each peak wavelength and their ratios. (A) and (B) are the dye in a buffer solution, and (C) and (D) are in nanoreactors.

FIG. 5A-5D are graphs of the pH dependent emission spectra of cSNARF-1 in the human plasma excited at 514 nm and the change of intensity at each given wavelength and their ratios. (A) and (B) are cSNARF-1 in the plasma, and (C) and (D) in nanoreactors.

FIG. 6A-6D are graphs of the pH dependent emission spectra of cSNARF-1 excited at 514 nm and the change of intensity at each given wavelength and their ratios. (A) cSNARF-1 in 3% albumin solution, B) cSNARF-1 in nanoreactors in 3% albumin solution, (C) cSNARF-1 in 1.5% IgG solution (D) cSNARF-1 in nanoreactors in 1.5% IgG solution. The iso-emission point of albumin is lost in (A), but it is regained once it is encapsulated. Despite a high concentration of IgG, the isoemission point is present in (C), as well as its encapsulated form in (D).

FIG. 7 is a graph showing an average of three stopped flow kinetic traces in response to pH change from pH 10 to 5 for cSNARF-1 in solution (open circles) and in nanoreactors (dark circles) are shown. A change in fluorescence intensity (67%) in solution took 175 msec, while that in nanoshells 125 msec, but the difference was considered within the range of errors.

FIG. 8A is a graph of relative fluorescence intensity at the red-most emission peak for c-SNARF-1 in DI water (solid circles) and in nanoreactors (open circles). Samples were exposed to a 300 W Xenon lamp (1.7×10 7 lux/m2) positioned 15 cm away from the sample. Sample temperature was controlled at 30 oC using a water circulating spectro-photometric cuvette holder.

FIG. 8B is a graph of intensity versus cycles for a test of reversible response of emission at 582 nm during pH cycling (open circles pH 9, closed circles pH 8, pH 6 crosses, pH 4 solid triangles). The emission ratio value is repeatable for each cycle.

FIGS. 9A and 9B are fluorescence microscope images of nanoreactors sandwiched between two coverglasses trapping a drop of Carboxy-SNARF-1 solution prepared at A) pH 4 and B) pH 9. Specimens were illuminated using a 490 nm excitation filter and emissions (shown above the images) collected after filtering out the primary excitation light.

FIG. 10 are time lapse fluorescence microscope images of Carboxy-SNARF-1 nanoreactors sandwiched between two cover glasses with a drop of pH 9 phosphate buffer between. Specimens were continuously illuminated through a 50× long working distance objective (Olympus) using a 490 nm excitation filter and emissions to the red of 520 nm were collected after filtering out the primary excitation light. The light intensity at the illuminated spot was 1.04*10⁹ lux/m².

### DETAILED DESCRIPTION OF EXEMPLARY EMBODIMENTS

The following detailed description of exemplary embodiments of the invention makes reference to the accompanying drawings, which form a part hereof and in which are shown, by way of illustration, exemplary embodiments in which the invention may be practiced. While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

The following detailed description and exemplary embodiments of the invention will be best understood by reference to the accompanying drawings, wherein the elements and features of the invention are designated by numerals throughout.

Definitions

In describing and claiming the present invention, the following terminology will be used.

The singular forms “a,” “an,” and “the” include plural referents unless the context clearly dictates otherwise. Thus, for example, reference to “a dye” includes reference to one or more of such materials and reference to “measuring” refers to one or more such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

pH Sensitive Nanoreactors

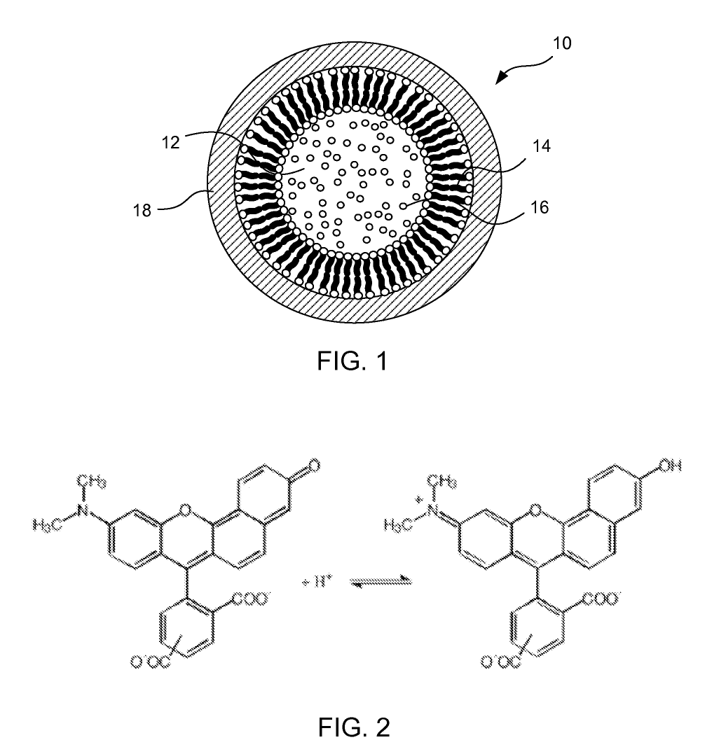

A pH sensitive nanoreactor 10 can include an aqueous core 12 within a liposome 14 as illustrated in FIG. 1. The aqueous core can include a pH responsive dye 16 dispersed or dissolved within the core. The liposome provides a nanoscale environment for the dye. Further, a nanoshell 18 can be present which encapsulates the liposome. The nanoshell can be permeable to hydrogen ions while also protecting the dye from exposure to deleterious compounds and photobleaching.

A number of pH responsive dyes can be suitable. Generally, such dyes are soluble in the aqueous core. Further, in order to provide an accurate measure of pH a dual-wavelength emission dye can be useful. However, single wavelength emission dyes can also be used. When dual color dyes are used, a ratio of two intensities can be obtained that are independent of concentration so that the measurement is absolute. Calibration of the particular dye can depend on the environment in which it is used and the lapsed time of use, as well as the intrinsic dye properties. Therefore, calibration is readily achieved once the particular nanoreactor design and materials are chosen. The particular emission wavelengths and associated dyes can have different pH sensitivities at various pH values. Therefore, the intended use can be at least partially designed and matched to a particular pH responsive dye.

Most useful for localized measurements because of their detection sensitivity as pH responsive dyes are fluorescent dyes. Non-limiting examples of suitable pH responsive dyes include carboxy-seminaphtorhodafluor-1 (carboxy-SNARF-1), seminaphthofluorescein, SNARF-5F carboxylic acid, SNARF-4F carboxylic acid, and others in the classes of carboxyseminapthorhodafluors (carboxy-SNARFs) and carboxy seminaphthofluoresceins (SNAFLs), and derivatives of fluorescein, anthracene, pyrene or quinone with single wavelength fluorescence emission and an absorption spectrum that changes with pH, and combinations thereof. These derivatives are known and can be readily obtained, for example, by addition of electron donating groups ortho to titratable phenol groups, reaction with carboxy groups, addition of titratable functional groups in the macrocycle, or the like. FIG. 2 illustrates the change in structure for carboxy-SNARF-1 at pH 5 and pH 10 which allows for a good pH sensitivity at a pH range of 5-10. In aqueous solution, the major proton titration takes place near neutral pH, with the carboxyl group protonated at acidic pH as illustrated by FIG. 2. In a narrow range near neutral pH the ratio of the intensity of the two fluorescence emission peaks of cSNARF-1 is less affected than most other pH sensitive dyes even in the presence of proteins and other substances. Non-fluorescent pH responsive dyes can optionally be used such as, but not limited to, bromo-phenol blue or bromo-cresol green could be used for applications which do not require high local resolution or low concentration detection. Although concentration can vary depending on the application and the particular dye (e.g. based on response intensity etc), as a general guideline, the concentration of dye in the aqueous core can be from about 0.05 micromolar to about 10 millimolar. Desirable concentrations for individual dyes can vary based on dye properties (e.g. Forster radius, etc.). At high concentrations, dyes with large Forster radii can interfere with one another or dyes can suffer inner filter effects. Depending on the size of the aqueous core and the dye molecules, there can often be from about 5 to about 25 dye molecules present in the aqueous core, and in some cases about 10 dye molecules. In one alternative, the aqueous core can consist essentially of water and at least one dye. In one alternative, salts can be added to the aqueous core such as, but not limited to, sodium chloride, magnesium chloride, or other alkali or alkaline metal chlorides. Optionally, a combination of dyes can be used. With multiple dyes, one dye is chosen having a pH response, while a second dye does not have a pH response. A signal from the non-pH responsive dye can be used to calibrate the first dye responses using the ratio of the first to second dyes within the aqueous core.

The liposome can typically be formed of a phospholipid. Suitable phospholipids can include, but are not limited to, L-α-phosphatidylcholine, phosphatidylethanolamine, phosphatidylglycerol, phosphatidylserine, phosphatidylinositol, sphingomyelin, dioleoyl phosphatidylethanolamine and combinations thereof. L-α-phosphatidylcholine is one particularly effective phospholipid. Other lipids can also be suitable as long as the packing factor allows formation of a liposome bilayer structure. The liposomes may be positively or negatively charged or net neutral. In either case, the phase stability of the mixture of lipids is such that under conditions of high ionic strength when the stabilizing shell-making precursors are added they do not disassemble. This can be unpredictable, but in general the more stable it is alone, the more likely it will be to be stable during the shell making process. For room temperature synthesis the melting point of the liposome regardless of its composition can typically be above room temperature. This melting point factor contributes to stability of the unit during processing. If an extremely high melting temperature (e.g. above about 60° C.) is chosen the suspension can become highly viscous and can be difficult, but not impossible, to reduce the phospholipid layers to liposome size using extrusion or sonication. Also, the driving force toward the equilibrium size/structure will be very strong and so the size transiently created by these methods will want to change back to the equilibrium structure.

However, other less common non-phospholipids (e.g. membrane mimetic amphiphiles) can also be used such as, but not limited to, those formed of dioxyethylene cetyl ether, cholesterol, oleic acid, ethoxylated fatty acids, fatty esters, palmitic acid, and the like (available as Novasomes or via synthetic routes known to those skilled in the art). In some cases such materials can require additives to form bilayers. For example, cholesterol does not form bilayers alone, and is used as an additive to stabilize other lipids. Other molecules which have only a single hydrophobic tail can also be similarly used. These materials can be optionally integrated with the phospholipids listed above to generate layers with tailorable transport, heat and ion sensitive properties.

The nanoshell can be formed of a material which is permeable to hydrogen ions but substantially impermeable to other species which would interfere with pH measurement. Suitable nanoshell materials can include calcium phosphate, silicate phosphates, silicate, mesoporous silicate and calcium phosphates, aluminum oxide, titanium oxide, magnesium oxide, and combinations thereof. Other metal-oxides which form at low temperatures via a precipitation process, without the use of harsh pH (in order to preserve the functionality of any encapsulated species) can also be used. Polymers like polyethylene glycol, polylactic-co-glycolic acids, and other polyionic polymers can also be used. Although the shell thickness can vary, typically a thickness from about 2 nm to about 10 nm is suitable. In one alternative, the shell surface can be free of polymer coatings. In another alternative, the nanoshell can be formed of a biodegradable material. Non-limiting examples of biodegradable materials include calcium phosphates, calcium carbonates, transition metal-doped calcium phosphates or carbonates, and combinations thereof.

The nanoreactors can generally be formed having a nanosize, e.g. less than 1 μm and often less than about 500 nm, although other sizes can also be formed based on the liposome materials and processing chosen. In one aspect, the nanoreactor can have a liposome diameter from about 90 nm to about 110 nm, although 20 nm to about 500 nm can broadly also be useful for pH detection.

These nanoreactors particularly allow for high pH sensitivity for relatively accurate pH measurement. Generally, these designs can allow a pH sensitivity of at least about 0.1 pH units, although up to 0.05, or even 0.01 pH unit sensitivity, can also be readily achieved.

Although not entirely understood, the protective nanoshell and liposome surrounding the pH sensitive dye reduce or prevent exposure of the dye to oxidizing via other chemical species (e.g. soluble quenchers, enzymes, etc.) and reduce potential electrical damage. Further, the dyes can be protected from spectral shifts caused by media components. Regardless of the underlying mechanism, the nanoreactors generally have a substantial photobleaching resistance. For example, in some cases like fluorescein dye a typical photobleaching of 50% in 2 hours under illumination with a 300 W xenon arc lamp is reduced to 0%. As a general guideline, photobleaching resistance can be dramatically increased over the free dye. In one aspect, photobleaching can be less than 5% over a 4 hour illumination with a 300 W xenon lamp.

The pH sensitive nanoreactors can be particularly suitable in measuring nanoscale pH in biological systems. In order to target specific tissues, organs or other areas, the nanoreactors can be injected locally and/or systemically administered. Further, the nanoreactors can be optionally functionalized on an exterior surface of the nanoshell with a group which selectively binds to a particular type of protein or other groups. For example, the functional group can be an antibody, chelating agent, or a reactable moiety like —SH or COOH which can be cross-linked to other molecules using commercially available conjugating reagents. Specific non-limiting examples of common antibodies which can be functionalized onto the nanoreactors can include IgA, IgD, IgE, IgG, IgM and combinations of these. Specific non-limiting examples of chelating agents include EGTA and organometallic molecules with exchangeable ligands.

Although other methods can be suitable, one approach to forming these nanoreactor pH sensors can include mixing an aqueous solution of the pH sensitive dye with a liposome-forming lipid. This mixture can then be extruded through an extrusion membrane to form a liposome suspension. The membrane pore size generally corresponds to the liposome diameter such that various size nanoreactors can be formed. Non-encapsulated dye and/or excess lipids can be removed via dialyzing or other suitable separation processes. Buffers can be added before and after extrusion in order to adjust pH to a desired level (e.g. pH 7 after formation).

The nanoshell can then be formed by ionic supersaturation of the solvent in the liposome suspension. Ionizable salts (e.g. calcium chloride and sodium phosphate) are added in small amounts to the suspension sufficient to collect around the periphery of the liposome which is charged. When the interaction around the periphery is sufficiently strong, the liposome changes structure which is undesirable. However, if the interaction is intermediate in strength then the local ion concentration rises causing precipitation of salts to form a thin layer of anionic solids to form the nanoshell.

The pH sensitive nanoreactors described herein can be particularly effective in measuring pH of nanoscale environments. The pH sensitive nanoreactor can be delivered or otherwise exposed to a nanoscale environment. The nanoscale environment can be almost any environment while the nanoreactor is capable of sensing pH changes at the nanoscale at least largely due to its size. The nanoshell can create a stronger pointed light source visible with a regular fluorescent microscope. One particular application includes measuring pH in an intracellular nanoscale environment. Such applications can be beneficial for measuring pH as a marker for disease, metabolism, drug response, etc. A calcium phosphate coating on the nanoshells is also biocompatible, so the nanoshells are suited for both in vitro and in vivo use. For example, pH can change the outcome of chemical reactions, such that it can cause breakdown of chemicals in microfluidic systems and it is a cellular marker for the onset of cancer. Alternatively, the nanoscale environment can be channels or volumes in a microfluidic device. Other applications include confocal and conventional fluorescence microscopy, near field scanning optical microscopy, and the like. For example, these nanoreactors can be used as pH responsive point sources in high-resolution imaging, stimulated emission depletion (STED) microscopy and photoactivated localization microscopy (PALM), which are used to increase the spatial resolution and sensitivity of microscopy measurements to 2-25 nm in size, a resolution improvement of 3-6 times over a confocal laser scanning microscopy.

An emission response of the pH responsive dye can be measured in any number of techniques including measuring emission intensity using a spectrophotometer. The emission intensity can then be correlated to a pH via a predetermined calibration scale. For example, fluorescein has a fluorescence intensity at 520 nm which changes when pH changes. The solution pH can be determined by looking for the corresponding fluorescence intensity. C. SNARF-1 dye is another dye which has a pKa of 7.5 at room temperature. It shows a significant pH dependent emission shift from yellow-orange to deep red fluorescence under acidic and basic conditions, respectively. This pH dependence allows the ratio of the fluorescence intensities from the dye at two emission wavelengths, typically 580 nm and 640 nm, to be used for quantitative determinations of pH. The nanoreactors of the present invention also have a high rate of kinetic response to pH changes (e.g. in the millisecond range such as around 200 ms). The pH sensitivity is also fully reversible.

### Example

A Carboxy-SNARF-1 dye filled nanoreactor with 100 nm in diameter is described. Nanoreactors is a general term referring to an enclosing structure that can serve as a host for a reaction and in some special cases can protect and/or enhance the reaction above that observed in dilute solution. In this example, nanoreactors are constructed from calcium phosphate stabilized egg phosphatidylcholine liposomes using procedures outlined below. The structure was characterized with the electron microscopy, dynamic light scattering, absorption spectroscopy, continuous and stopped flow fluorimetric measurements. The calcium phosphate based nanoreactor capsule creates an environment for the carboxy SNARF-1 in which it is able to resist photobleaching and quenching problems and is capable of measuring pH via dual wavelength emission ratios in the physiological range of pH, with a resolution of about 0.05 pH units, a response time of about 10 ms, and resistance to quenching by outside chromophores.

Synthesis

The pH responsive 5-(and-6)-Carboxy-seminaphthorhodafluor-1 (Carboxy-SNARF-1) was obtained from Invitrogen (Carlsbad, Calif.). Sodium phosphate monobasic (NaH₂PO₄.H₂O), dibasic sodium phosphate (Na₂HPO₄.H₂O), and calcium chloride (CaCl₂) were obtained from Fisher Scientific (Waltham, Mass.). Carboxyethyl phosphonic acid (CEPA) was obtained from Sigma-Aldrich (St. Louis, Mo.). L-a-Phosphatidylcholine (EPC) was obtained from Avanti Lipids (Alabaster, Ala.). All solutions were prepared in E-pure water with a resistivity of 18.2 MΩ-cm obtained from a Barnstead E-Pure

(Barnstead/Thermolyne, Dubuque, Iowa) ultra-pure water system. Polycarbonate 25 mm filters with 100 nm pore size were obtained from Fisher Scientific (Waltham, Mass.). Spectra/Por dialysis membrane tubing with a MWCO of 3,000 and 12-14,000 Daltons and PM30 30,000 MWCO ultrafiltration membranes were obtained from VWR Scientific (West Chester, Pa.). Human plasma was purchased from ARUP Laboratories (Salt Lake City, Utah).

To produce c-SNARF-1 loaded EPC liposomes, 2 mg of cSNARF-1 solid was mixed with 18 ml of pH 9.5 phosphate buffer prepared by adjusting 200 mM dibasic sodium phosphate to pH 9.5. Twenty-five mg of EPC lipid was air-dried at room temperature to remove chloroform, and the residue hydrated in 5 ml of the cSNARF-1 solution prepared in the above. The final concentration of cSNARF-1 was 0.25 mM and that of EPC was 0.64 mM. The mixture was magnetically stirred at 800 RPM for 30 minutes at room temperature in a 25 ml beaker. The mixture was extruded 10 times through a 25 mm diameter 100 nm pore size Millipore polycarbonate extrusion filter using a 10 ml Thermobarrel LIPEX extruder (Northern Lipids, Burnaby, BC, Canada). The resulting liposome suspension was left undisturbed for 1 hour.

To coat calcium and phosphate over the lipsomes, first, 1.32 ml of 0.1 M calcium chloride solution were added to 10 ml of DI water and pH adjusted to 8.0. The solution was stirred continuously at 800 RPM, and 3.6 ml of the previously prepared liposome suspension were added incrementally in 0.2 ml portions every hour over the course of 18 hours. Since the liposome suspension was prepared in phosphate buffer at pH 9.5, the addition of liposomes to the calcium chloride solution raised the pH and initiated the reaction of calcium with phosphate. The net result was the formation of a thin mineral shell around the liposome. At the end of the liposome titration, 1.2 ml of 0.1 M CEPA at pH 7 were added to the suspension in order to carboxylate the coating surface and stirring continued for an additional 2 hours. To remove un-reacted reagents, the suspension was dialyzed against phosphate buffer at pH 8.

This procedure produced a nanoreactor suspension having the overall dye concentration of 0.2 M and a calculated particle concentration of 20 nM assuming that the entire lipids were used in the formation of liposomes. The suspension was concentrated using an Amicon Series 8000 Stirred Cell with 30,000 MWCO ultrafiltration membrane to a concentration of approximately 1.5 M of particles and 15 M in dye, which is equivalent to 10 dyes per particle.

Physical Characterization

The mean particle hydrodynamic diameter of the product was obtained using a Zetasizer Nano ZEN3600 (Malvern Instruments, Malvern, Worcestershire, UK). The typical polydispersity of the suspension was 0.261. Transmission electron microscope (TEM) images of the particles were obtained using a Tecnai T12 TEM electron microscope (Philips, Andover, Mass.) operated at 100 KV. For this, a 2 μl of each specimen was placed on a 300 mesh Formvar-coated carbon grid (Ted Pella, Redding, Calif.) and dried at room temperature.

Absorption and Fluorescence Spectrophotometry

Absorption spectra were obtained using a UV Mini 1240 spectrophotometer (Shimadzu Scientific Instruments, Columbia, Md.) in a 1-cm path length quartz cuvette. The total concentration of encapsulated cSNARF-1 in the suspensions was determined at pH 7 using the extinction coefficient of 27,000 L-mol-1-cm-1 at the absorbance maximum of 548 nm. Fluorescence spectra were obtained using a Cary Eclipse fluorescence spectrophotometer (Varian, Walnut Creek, Calif.) with excitation wavelength of 514 nm and collecting emissions over the range of 550 nm to 750 nm at room temperature. The pH-dependent fluorescence spectra of cSNARF-1 solution and particle suspensions were obtained in water, plasma, 3% albumin solution and 1.5% IgG solution. The concentrations of albumin and IgG were chosen to be close to the concentrations found in the human plasma. The pH was adjusted using 0.1 N HCl or NaOH, starting from pH 7 to either the higher or lower limit of pH and back. All spectra were corrected for dilution.

Stopped Flow Kinetics

The time dependent response of cSNARF-1 solution and nanoreactors to the pH change was measured at 20° C. using an RX2000 Stopped-Flow Mixing Accessory (Applied Photophysics Limited, Leatherhead, UK) attached to the Cary Eclipse fluorescence spectrophotometer. The mixing time of this system is reported to be 8 msec, and the response time of Eclipse is 1 msec. To estimate the response time of the nanoreactors, they were first adjusted to pH 10 then mixed with a 50 mM phosphate buffer solution at pH 2. By exciting cSNARF-1 at 514 nm during this process, the pH-responsive fluorescence change at 582 nm was recorded.

Photobleaching

cSNARF-1 in DI water at pH 9 and the nanoreactors in the same solvent was placed into covered 1 cm-path length quartz cuvettes and kept at 30° C. using a circulating water bath while being continuously exposed to the emission of a 300 W Xenon lamp placed 15 cm away. The light intensity at the sample was measured using a DX-200 digital illumination meter (Edmund Optics, Barrington, N.J.) and it was found to be 1.69×10⁷ lux/m². The fluorescence intensity at 640 nm was recorded at 5 or 10 minute intervals.

Fluorescence Microscopy

An Olympus IX71 fluorescence microscope (Olympus America Inc., Melville, N.Y.) with Qimaging Retiga 1300 color CCD camera (Quantitative Imaging Corporation, Burnaby, BC, Canada) was used to visualize the cSNARF-1 nanoreactors at the two extreme pH values. In another experiment, a sample at pH 9 was observed over the course of 80 minutes of continuous microscopic observation. During the course of the latter experiment, the edge of coverslip was sealed with Eukitt mounting medium (Calibrated Instruments, Inc., Hawthorne, N.Y.). The microscope images were usually taken at an exposure time of 200 ms and 500× magnification. The light intensity at the point of illumination was 1.04×10⁹ lux/m².

Particle Characterization

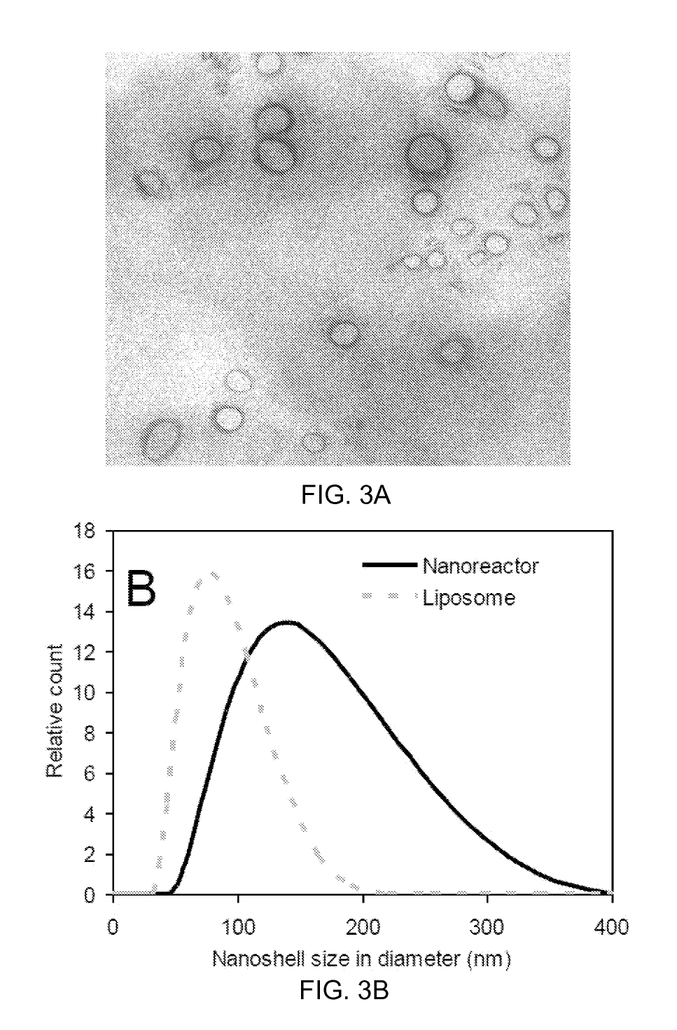

A transmission electron microscope (TEM) image of cSNARF-1 filled nanoreactors is shown in FIG. 3A. The hollow nanoreactors appear to be surrounded with a definable mineral shell. The mean size of EPC liposomes used to prepare nanoreactors and that of the cSNARF-1 nanoreactors were 100 and 150 nm, respectively (FIG. 3B), and the distribution of their sizes at 50% of each peak are ±30 nm and ±70 nm, respectively.

Measuring the Carboxy SNARF-1 Content in Nanoshells Using Absorption Spectroscopy.

The overall concentration of Carboxy-SNARF-1 in a pH 9 suspension containing 1.1 μM of particles was found to be 1.5×10⁻⁵M, and the encapsulated concentration was calculated to be approximately 3.89×10⁻⁵M by assuming all particles were identical in size and capacity. This corresponds to ˜13 Carboxy-SNARF-1 molecules in each nanoshell particle. The encapsulation efficiency is about 52.5% as estimated by taking the ratio of the overall Carboxy-SNARF-1 concentration in nanoreactors suspension after dialysis to the concentration before dialysis.

SNARF Dye pH Dependent Emission Properties in Solution, DOPA Liposomes, and Nanoshells.

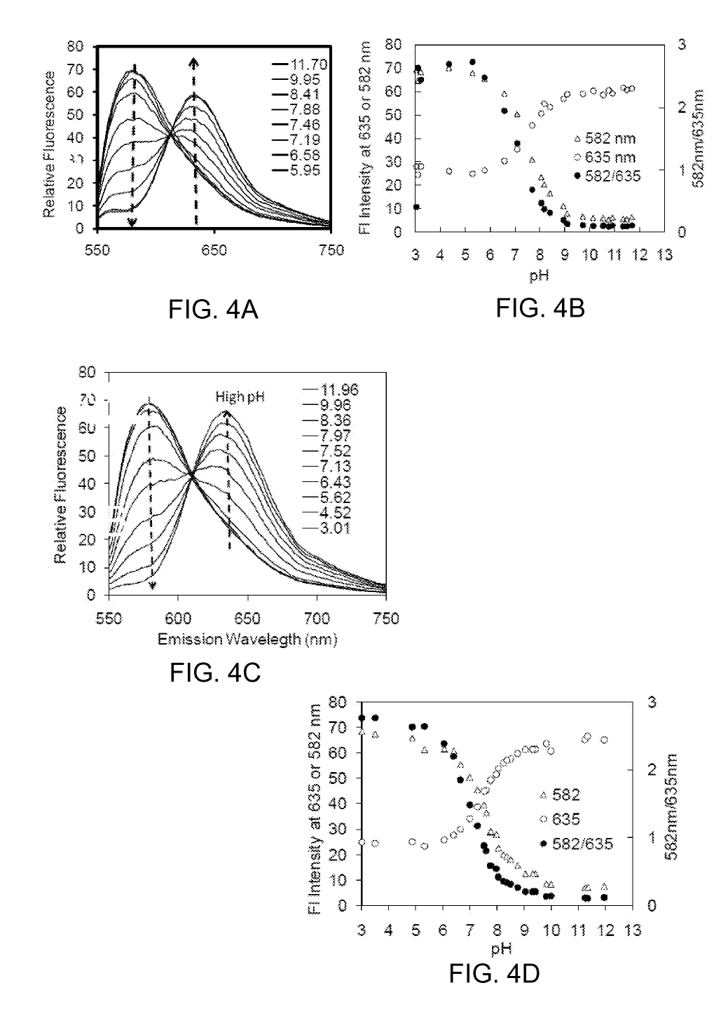

The pH dependent emission spectra ranging from pH 3 to 12 for 4.5 M cSNARF-1 in water are shown (FIG. 4A). The direction of change in intensity for each of the two peak wavelengths of cSNARF-1 as the pH increased is indicated with an arrow. At 625 nm, the intensity is increased as the pH increased, but at 582 nm it is decreased.

A more quantitative analyses of these changes in fluorescence intensity may be carried out using the modified Henderson-Hasselbalch equation. For a dye with dual wavelength emissions, the ratio of fluorescence intensity over a given pH range elucidates the dye's pKa as:

$${pK}_{a} = {pH}_{i} - c log ( \frac{{R}_{i} - {R}_{min}}{{R}_{max} - {R}_{i}} ) - log ( \frac{I ( A )}{I ( B )} ) , \qquad ( 1 )$$

where Rᵢ is the fluorescence intensity ratio of the two wavelengths (582 nm and 635 nm) at pHᵢ, Rₘᵢₙ and Rₘₐₓ are the minimum and maximum limiting values of Rᵢ, respectively. The value of I(A)/I(B) is obtained from the emission intensities at 635 nm for the limiting acidic, I(A), and basic, I(B), pH regions. In practice, to elucidate the pKₐ of each sample, first the pH_{1/2} value, at which (Rᵢ−Rₘᵢₙ)=(Rₘₐₓ−Rᵢ) or R_{1/2}=(½) (Rₘₐₓ+Rₘᵢₙ) was determined to make the second term of the right hand side of equation equal to zero. The value of pH_{1/2} was then subtracted by log(I(A)/I(B)) to estimate the pKa value.

To estimate the pH_{1/2} and I(A)/I(B) from the experiment, the fluorescence intensity changes at 635 nm and 582 nm, and their ratios were plotted against the pH values as shown in FIG. 4B. For elucidation of both pH_{1/2} and I(A)/I(B), it is important to note that well defined limiting intensities at both extreme ends of acidic and basic pHs must be available. Furthermore, the spectral changes shown in each of FIG. 4A should have a well defined isoemissive point to assure that the integrity of spectral chromophores is maintained throughout the entire pH range. Once pH_{1/2}, Rₘᵢₙ, Rₘₐₓ, and I(A)/I(B) are determined, the value for “c” may be estimated from the least squares fit of the equation to the experimental results shown as filled circles in FIG. 4B. The results are summarized in Table I.

TABLE I

| cSNARF-1 in | Solvent | pH 1/2 | Acidic peak | Basic peak | log{I[A]/I[B]} | pKa | c |
|---|---|---|---|---|---|---|---|
| Water | Water | 7.15 | 581 nm | 633 nm | −0.31 | 7.46 | −1.17 |
| Nanoreactor | Water | 7.05 | 582 nm | 635 nm | −0.43 | 7.48 | −1.15 |
| Water | Plasma | ? | ? | ? | ? | ? | ? |
| Nanoreactor | Plasma | 7.30 | 592 nm | 638 nm | −0.32 | 7.62 | −1.06 |
| Water | 3.0% Albumin sln | ? | ? | ? | ? | ? | ? |
| Manoreactor | 3.0% Albumin sln | 7.30 | 585 nm | 638 nm | −0.28 | 7.58 | −1.22 |
| Water | 1.5% IgG sln | 7.25 | 584 nm | 633 nm | −0.47 | 7.72 | −0.78 |
| Nanoreactor | 1.5% IgG sln | 7.30 | 582 nm | 633 nm | −0.37 | 7.67 | −0.84 |
| Mean | | 7.23 | | | −0.36 | 7.59 | −1.04 |
| S.D. | | 0.10 | | | 0.07 | 0.10 | 0.18 |

The pHᵢ, of a solution, where cSNARF-1 is present, may thus be estimated by substituting the experimentally determined pKa, Ri, Rmin, Rmax, and log(I(A)/I(B)) to Equation 1. It is important to note that if any of these parameters cannot be defined, the pH value of the solution may not be estimated based on Equation 1.

To test if encapsulation cSNARF-1 in nanoreactors would affect the pH determination by cSNARF-1 or not, a similar fluorometric analysis was performed with cSNARF-1 nanoreactors, and the results are shown in FIG. 4C and FIG. 4D. The parameters equivalent to what have been described in the above are obtained and the results are also shown in Table 1. The result demonstrates that fluorescence spectral responses of cSNARF-1 in solution and in nanoreactors are similar within the range of experimental errors.

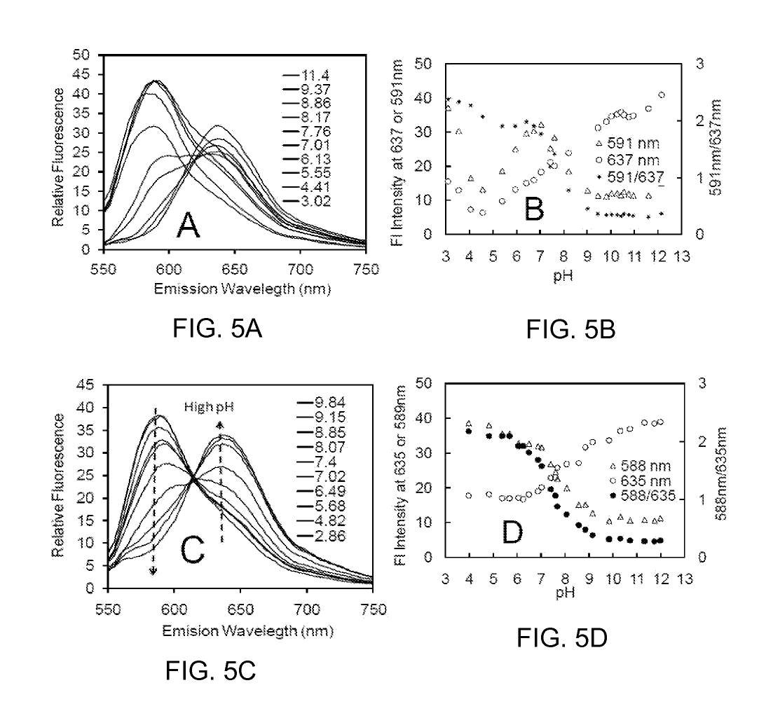

To test if the nanoreactor can protect encapsulated cSNARF-1 from the solutes outside of the nanoreactor, similar experiments were carried out for human plasma, 3% albumin, and 1.5% IgG solutions. The pH dependent fluorescence spectra of cSNARF-1 in human plasma (FIG. 5A), and change in the magnitude of fluorescence peaks at 591 nm and 637 nm, as well as their ratios are plotted (FIG. 5B). FIG. 5A clearly demonstrates lack of isoemissive point, and in FIG. 5B, there is a dip in fluorescence intensity of 591 nm at the acidic range of pH making it difficult to define the limiting intensity. In fact, the difficulty of defining limiting fluorescence intensity persists at pH extremes for both wavelengths. As a consequence, the data cannot be used to establish necessary parameters needed for Equation 1 and so stated in Table I. On the other hand, similar analyses for cSNARF-1 encapsulated as nanoreactors and suspended in the plasma (FIGS. 5C and 5D) demonstrate that elucidation of the parameters needed for Equation 1 is plausible and the results are summarized in Table I. The results are similar to those of cSNARF-1 in solution and strongly suggest that the pH, of the plasma can be estimated using the cSNARF-1 nanoreactors.

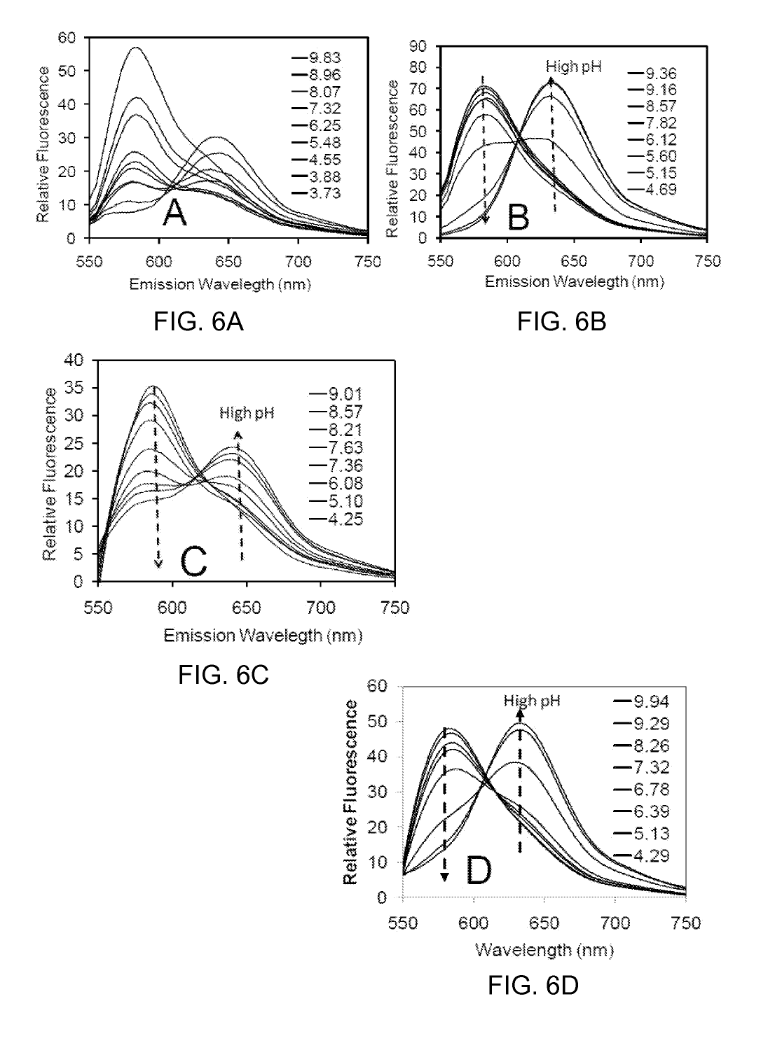

The loss of the isoemissive point, disturbance in pH titration of the fluorescence spectra, and the fact that these effects are reduced by encapsulating the dye in nanoreactors suggest possible interaction of the ions and molecules(s) in the plasma with the dye. If ionic interaction is the cause of such interference, it may be assumed that these interactions occur at those pH region(s), where cSNARF-1 and the binding molecules have opposing charges. Therefore, it may be hypothesized that an acidic protein, such as albumin which is a major component of plasma (found at 3%) with pKₐ=4.7, could interact with cSNARF-1 in a given pH region. The pH dependent fluorescence spectra of cSNARF-1 in 3% albumin solution (FIG. 6A) are similar to what was observed in plasma, while that of cSNARF-1 nanoreactor in 3.0% albumin is more like the case of pure water and is shown in FIG. 6B. From such results the parameters needed for Equation 1 may be elucidated and the results are also shown in Table I for comparison, showing a good agreement with those of cSNARF-1 in solution. In contrast, the pH dependent fluorescence spectra of cSNARF-1 in solution containing 1.5% IgG, with aver pI 6.95, are shown in FIG. 6C which indicates little interaction between them. All the parameters needed for Equation 1 can be elucidated and shown in Table I with only a slight increase in pKa. Using the cSNARF-1 nanoreactor in 1.5% IgG, the pH dependent fluorescence spectra are similar to that of cSNARF-1 in solution and the pKa value becomes slightly closer to that of cSNARF-1 in water. These results indicate that the encapsulation protects the dye from the external molecules.

The Rate of Proton Transport into Nanoreactors

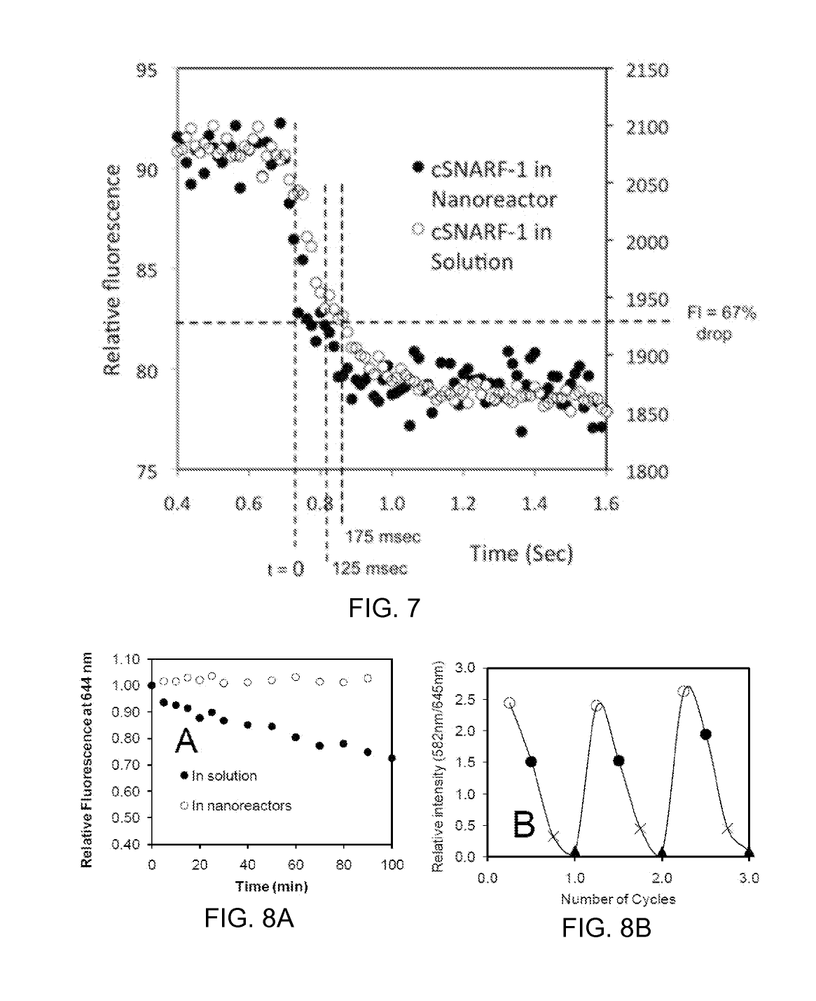

The average of three stopped flow kinetic traces in response to pH change for cSNARF-1 in solution (open circles) and in nanoreactors (closed circles) are shown in FIG. 7. A 65% change in fluorescence intensity (t65%) in solution took 175 msec, while that in nanoshells 125 msec, but the difference was within the range of error. Therefore, it may be concluded that the time response of encapsulated dye is almost as fast as that in solution.

Photobleaching and pH Reversibility Test

An increased resistance to photobleaching of cSNARF-1 nanoreactors (open circles) compared with that of the dye in solution (filled circles) is evident in FIG. 8A. The loss of fluorescence intensity of cSNARF-1 in aqueous solution is as large as 30% in solution after 100 minutes, but in the nanoreactors the loss is negligible. Both the absorbance and fluorescence emission spectra of the samples were the same before and after illumination (not shown). The cause of resistance to photobleaching in the nanoreactors may be attributed to many factors, but the usefulness of bleach-resistant fluorescence is evident in many time-dependent studies.

The reversibility of fluorescence intensity and the ratio R of cSNARF-1 nanoreactor suspension under cyclic change of pH change from pH 6.0, to 10.0 was confirmed in FIG. 8B.

Fluorescence Microscopic Images

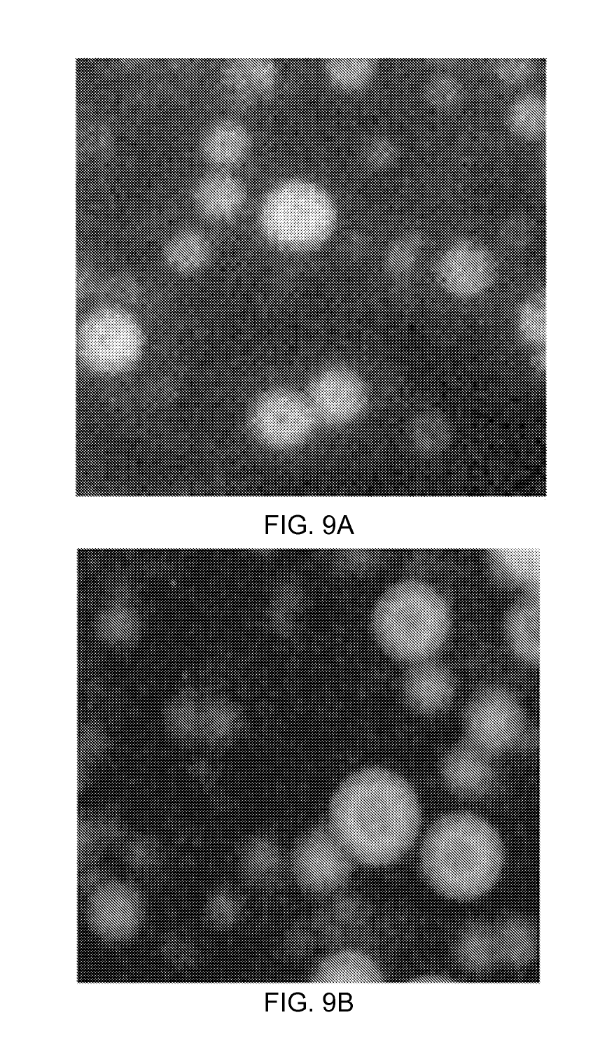

Even though the size of the cSNARF-1 filled nanoreactor is close to the resolution of an optical microscope (˜0.2 m), the fluorescent particles may still be seen with a fluorescent microscope as long as its fluorescence intensity strong enough and particles are separated by distances greater than the optical limit of resolution. The fluorescent images of cSNARF-1 in nanoreactors excited at 490 nm at pH 4 (FIG. 9A) and 9 (FIG. 9B) are shown. Yellow-green and orange fluorescence emissions reflect the difference in pH values and the size differences reflect the distribution of the particle sizes observed with EM and dynamic light scattering studies (FIGS. 3A and B), with some distortions caused by aggregation, and variations in focal plane relative to the particle location. Because the emission of the pH 9 sample is red with respect to the emission from the pH 4 specimen, its resolution appears slightly reduced.

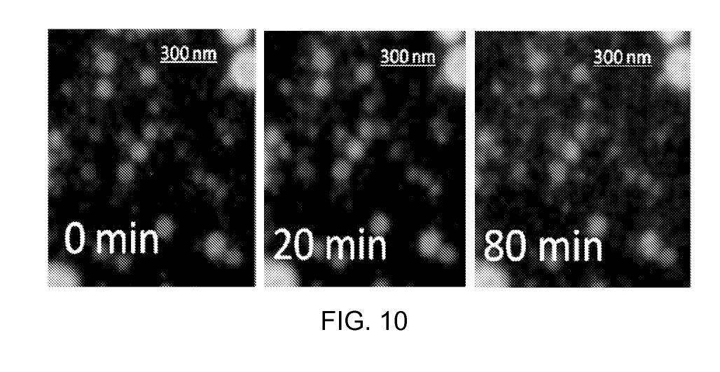

Prolonged and continuous observation with the fluorescence microscopic can lead to photobleaching and thermal degradation of nanoreactors. In FIG. 10 shown are the time-lapse images of nanoreactors at 488 nm. After about 80 minutes of exposure the integrity of the particle appears to be compromised.

Discussion

Once calibrated, the dual wavelength fluorescent pH sensors make it possible to estimate the pH value at their location without knowing their concentrations by using the fluorescence ratio at two wavelengths described as shown in Equation 1. Although Equation 1 is valid for dual wavelength fluorescent dyes under optimal conditions, there is a limitation evident when the dye is exposed to a high concentration of molecules, as is the case in the living system, and especially when acidic proteins are present. Once the dye is properly isolated from the surrounding via a proton permeable membrane, its function as a pH sensor can be upheld even in the presence of interfering molecules.

It is shown that in plasma and in albumin solution, the pH dependent fluorescence spectra of cSNARF-1 loses its isoemissive point, as well as the well-defined limiting fluorescence intensity ratios, especially in the acidic region. As a consequence, measurements of pH using cSNARF-1 (without the nanoreactor configuration) cannot be carried out with the application of Equation 1. Preventing the molecular interaction between cSNARF-1 and its counterpart, such as albumin benefits from an effective shield between them that does not interfere with the pH function of cSNARF-1. Tested in this example is a layer of liposome coated with a layer of self-assembled brushite. The results demonstrate that the liposome interrupts the interaction between cSNARF-1 with solutes outside the shell restoring the isoemmissive point and the applicability of Equation 1 for analysis of the pH dependent behavior of the dye without decreasing the rate of detection of pH changes.

Parameters used for Equation 1 were elucidated for cSNARF-1 in solution and in nanoreactors, and in different solutions. These parameters could not be elucidated when cSNARF-1 was directly dissolved in the plasma or albumin due to interaction effects, but they were deducible when cSNARF-1 nanoreactors were used. It is also important to note that each type of parameter among the various analyzable samples is similar with relatively small standard deviation.

The average size of cSNARF-1 containing nanoreactor seen by TEM and DLS is about 150 nm which is below the resolution of an optical microscope, but its fluorescence may be recognized with a conventional florescence microscope given the conditions described earlier, and despite the observation that the calculated dye content per particle is approximately ten. The colors of the nanoreactors prepared in pH 4 and 9 solutions were visually distinguishable, suggesting that the quantitative difference could be revealed using a microscopic spectrophotometer. The fact that the particle is optically recognizable with a relatively small number of dyes is advantageous, since the change of pH can be detected with the expense of only a small number of protons in the solution making it possible to use the nanoreactor as an optical sensor without interfering with nearby pH dependent processes.

The cSNARF-1 nanoreactors are more photobleaching resistant than the dye in solution and response to change in pH is reproducible over at least several cycles. The integrity of the nanoreactor was maintained up to 80 minutes of continuous illumination using a fluorescence microscope.

Thus, once dual wavelength pH sensitive dyes are made into a nanoreactor form, and the parameters for the modified form of the Henderson-Hasselbach equation for the dye in solution are elucidated, the pH values of a sample even in the presence of interfering molecules can be measured by experimentally determining the fluorescence ratio at the two wavelengths. Having a low dye molecule count in each nanoreactor on average, it is possible to use the nanoreactor as a pH nanosensor without disturbing the surrounding equilibrium.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A pH sensitive nanoreactor, comprising:
a) an aqueous core within a liposome, said aqueous core including a pH responsive dye; and
b) a nanoshell encapsulating the liposome, said nanoshell being permeable to hydrogen ions.

2. The nanoreactor of claim 1, wherein the pH responsive dye is a dual-wavelength emission dye.

3. The nanoreactor of claim 1, wherein the pH responsive dye is a fluorescent dye.

4. The nanoreactor of claim 1, wherein the pH responsive dye is selected from the group consisting of carboxy-seminaphtorhodafluor-1 (carboxy-SNARF-1), seminaphthofluorescein, SNARF-5F carboxylic acid, SNARF-4F carboxylic acid, carboxyseminapthorhodafluors (carboxy-SNARFs), carboxy seminaphthofluoresceins (SNAFLs), derivatives of fluorescein, anthracene, pyrene and quinone with single wavelength fluorescence emission and an absorption spectrum that changes with pH, and combinations thereof.

5. The nanoreactor of claim 4, wherein the pH responsive dye is carboxy-SNARF-1.

6. The nanoreactor of claim 1, wherein the pH responsive dye is a non-fluorescent dye which includes at least one of bromo-phenol blue and bromo-cresol green.

7. The nanoreactor of claim 1, wherein the liposome is formed of a phospho lipid.

8. The nanoreactor of claim 1, wherein the phospholipid is selected from the group consisting of L-α-phosphatidylcholine, phosphatidylethanolamine, phosphatidylglycerol, phosphatidylserine, phosphatidylinositol, and combinations thereof.

9. The nanoreactor of claim 7, wherein the phospholipid is L-α-phosphatidylcholine.

10. The nanoreactor of claim 1, wherein the nanoshell is formed of a member selected from the group consisting of calcium phosphate, silicate phosphates, silicate, mesoporous silicate, calcium phosphates, aluminum oxide, titanium oxide, magnesium oxide, and combinations thereof.

11. The nanoreactor of claim 9, wherein the nanoshell is formed of calcium phosphate.

12. The nanoreactor of claim 1, wherein the nanoreactor has a diameter from about 90 nm to about 110 nm.

13. The nanoreactor of claim 1, wherein the nanoreactor has a pH sensitivity of at least about 0.1 pH units.

14. The nanoreactor of claim 1, wherein the nanoreactor has a photobleaching resistance of about 0% over a 2 hour illumination with a 300 W xenon arc lamp.

15. The nanoreactor of claim 1, further comprising at least one of an antibody, a chelating agent, and a reactable moiety coated on an exterior surface of the nanoshell.

16. A method of measuring pH of nanoscale environments, comprising:
a) providing a pH sensitive nanoreactor, comprising:
i. an aqueous core within a liposome, said aqueous core including a pH responsive dye; and
ii. a nanoshell encapsulating the liposome, said nanoshell being permeable to hydrogen ions;
b) delivering the pH sensitive nanoreactor to the nanoscale environment; and
c) measuring an emission response of the pH responsive dye.

17. The method of claim 15, wherein the nanoscale environment is intracellular.

18. The method of claim 15, wherein the nanoscale environment is a micro fluidic device.

19. The method of claim 15, wherein the measuring the emission response includes measuring emission intensity and correlating with a solution pH.

20. The method of claim 17, wherein the measuring emission intensity uses a spectrophotometer.

21. The method of claim 15, further comprising functionalizing the nanoshell with an antibody.
